(19) United States
(12) Patent Application Publication
(10) Pub. No.: US 2025/0387298 A1
(43) Pub. Date: Dec. 25, 2025

(54) FLEXIBLE MEDICAL CONTAINER

(71) Applicant: B. Braun Melsungen AG, Melsungen (DE)

(72) Inventors: Shukun Wang, Camarillo, CA (US); Alex Ionkin, Kennett Square, PA (US); Ben Yu, Irvine, CA (US); Philippe Chanson, Préverenges (CH); Carlo Leonardi, Novara (IT); Huaina Li, San Clemente, CA (US); Rith Thuy-Boun, Irvine, CA (US); Krista Lumsden-Friedli, Reverolle (CH)

(21) Appl. No.: 18/750,453

(22) Filed: Jun. 21, 2024

(51) Int. Cl. A61J 1/10; A61K 31/431

(52) U.S. Cl. CPC A61J 1/10; A61K 31/431

(57) ABSTRACT

A flexible medical container has a wall that includes an inner layer. The inner layer includes a polypropylene material made in the presence of a metallocene catalyst.

### FIELD

The present disclosure relates to a flexible medical container.

### BACKGROUND

Solutions containing an active pharmaceutical ingredient (API) are commonly administered intravenously from flexible medical containers to patients.

During their storage, active pharmaceutical ingredients (APIs) interact with inner surfaces of the flexible medical containers. Such interactions, however, may induce undesired reactions such as aggregation, oligomerization and/or decomposition of the APIs, thereby fostering turbidity formation of solutions of APIs rendering them ineffective or unsuitable for administration.

### SUMMARY

The object underlying the present disclosure is therefore to make available a medical container for storage of active pharmaceutical ingredients, which at least partially avoids the above-mentioned drawbacks.

The present disclosure refers to a medical container, in particular a medical plastic container.

Preferably, the medical container is for storage of a solution, typically an aqueous solution, containing an active pharmaceutical ingredient. Particularly, the solution is to be administered intravenously.

Typically, the medical container is a flexible, i.e. pliable or soft, medical container, in particular a flexible medical plastic container.

Especially preferred, the medical container may be in the form of a medical bag.

The medical container comprises or has a wall comprising an inner layer, wherein the inner layer comprises a polypropylene material. Preferably, the polypropylene material is made, i.e. produced or synthesized, via a metallocene catalyst or in the presence of a metallocene catalyst.

The term “medical container” as used according to the present disclosure means a container, in particular rigid, semi-rigid or flexible container, which can be used in the field of medicine.

The term “medical plastic container” as used according to the present disclosure means a medical container comprising a plastic (in particular as detailed in the following description) or consisting of a plastic (in particular as detailed in the following description).

The term “inner layer” as used according to the present disclosure means a layer of the medical container that is configured or adapted to encase or surround, in particular directly, i.e. immediately, an active pharmaceutical ingredient and/or a liquid diluent, in particular a solution, preferably an aqueous solution, containing an active pharmaceutical ingredient.

The term “polypropylene material” as used according to the present disclosure means a material comprising polypropylene or consisting of polypropylene.

The term “polypropylene” as used according to the present disclosure may mean a polypropylene homopolymer and/or a polypropylene copolymer, in particular a polypropylene block copolymer, a polypropylene random copolymer or a polypropylene graft copolymer. Preferably, the term “polypropylene” as used according to the present disclosure means a polypropylene homopolymer.

The term “polypropylene homopolymer” as used according to the present disclosure means a polymer which is produced or synthesized by polymerization, in particular chain-growth polymerization, of propylene (propene). The polypropylene homopolymer or a polypropylene homopolymer block of a propylene copolymer may have an isotactic, a syndiotactic or an atactic structure.

The term “polypropylene copolymer” as used according to the present disclosure means a polymer which is produced or synthesized by polymerization, in particular chain-growth polymerization, of propene and at least one further sort or type of monomer such as ethene.

The term “metallocene catalyst” as used according to the present disclosure means an organometallic coordination compound in which one or two cyclopentadienyl rings or substituted cyclopentadienyl rings are 7-bonded to a central transition metal atom.

The term “active pharmaceutical ingredient” as used according to the present disclosure means any ingredient that provides biologically active other, in particular direct, effect in diagnosis, cure, mitigation, treatment or prevention of disease or that affects a structure or any function of a body of humans or animals. Further, the term “active pharmaceutical ingredient” may particularly mean an active pharmaceutical ingredient in its neutral form or a pharmaceutically acceptable salt of an active pharmaceutical ingredient.

The present disclosure rests on the overall surprising finding that turbidity formation in an aqueous solution of an active pharmaceutical ingredient may be significantly reduced if stored in a medical container according to the present disclosure in comparison to conventional medical containers. Therefore, the medical container according to the present disclosure advantageously contributes to a higher solubility, and thus higher stability of the active pharmaceutical ingredient resulting in a longer effectiveness of the active pharmaceutical ingredient for administration. In particular, the present disclosure is based on the surprising finding that due to the metallocene catalyst the quantity of oligomers (C2-C24) during the synthesis of polypropylene being capable of migrating easily into the solution due to their low size and being capable of forming aggregates and/or reacting with the solution can be strongly reduced.

In an embodiment of the present disclosure, the metallocene catalyst is a titanium-free metallocene catalyst, i.e. a catalyst containing no titanium, in particular not as a transition metal. In that regard, the inventors could demonstrate that titanium, in particular in the form of titanium containing compounds, has a substantial effect on turbidity formation. Without wishing to be bound by any theory, titanium may induce surface oxidation of the inner layer and change properties of the surface, for example increase surface area and/or roughness of the surface, thereby promoting undesired interactions with the active pharmaceutical ingredient such as aggregation, oligomerization (e.g. dimerization), coordination, and/or decomposition of the active pharmaceutical ingredient. Thus, by using a titanium-free metallocene catalyst for the production or synthesis of the polypropylene material, turbidity formation may be advantageously reduced.

More specifically, the metallocene catalyst preferably comprises zirconium as transition metal.

In a further embodiment of the present disclosure, the polypropylene material is free of titanium, in particular elemental titanium and/or titanium containing compounds. With respect to the titanium containing compounds, the titanium has typically the oxidation state +4. The advantages mentioned in the preceding paragraph do apply mutatis mutandis.

In a further embodiment of the present disclosure, the polypropylene material has an amount of aluminum, in particular elemental aluminum and/or aluminum containing compounds, <80 μg/g (i.e. <80 μg aluminum per g of the polypropylene material), preferably <60 μg/g (i.e. <60 μg aluminum per g of the polypropylene material), in particular <40 μg/g (i.e. <40 μg aluminum per g of the polypropylene material), in particular of 1 μg/g to 25 μg/g (i.e. 1 μg aluminum per g of the polypropylene material to 25 μg aluminum per g of the polypropylene material). With respect to the aluminum containing compounds, the aluminum has typically the oxidation state +3. The inventors could further demonstrate that also aluminum has a substantial effect on turbidity formation. In that regard, the turbidity formation may be caused by the same or similar processes as described in the context of titanium. Thus, advantageously, also a low amount of aluminum in the polypropylene material may contribute to a lower turbidity formation.

Further preferred, the polypropylene material may have a weight average molecular weight (Mw), in particular determined via gel permeation chromatography, of 170.000 to 250.000, in particular 190.000 to 230.000, preferably 200.000 to 210.000, for example, of 204.000.

Further preferred, the polypropylene material may have a number average molecular weight (Mn), in particular determined via gel permeation chromatography, of 40.000 to 100.000, in particular 60.000 to 90.000, preferably 70.000 to 80.000, for example, of 73.000.

In a further embodiment of the present disclosure, the polypropylene material has a polydispersity index (Mw/Mn) of 1 to 4, in particular 2 to 3, preferably 2.5 to 3, for example of 2.8. The polydispersity index calculated is the weight average molecular weight (Mw) divided by the number average molecular weight (Mn). The polydispersity index indicates the distribution of individual molecular masses in a batch of polymers. Thus, the polypropylene material advantageously has less diverse short-chain polypropylene molecules and/or long-chain polypropylene molecules or, in other words, a more homogeneous distribution of polymer chains. This may advantageously result in a smoother surface of the inner layer. A smoother surface of the inner layer in turn decreases undesired interactions between the active pharmaceutical ingredient and the surface of the inner layer. Thus, turbidity formation may also be significantly reduced.

In a further embodiment of the present disclosure, the polypropylene material is free of a filler, in particular silica, preferably synthetic silica. The term “filler” as used according to the present disclosure means a material being capable of improving properties, for example tensile strength, toughness, heat resistance, colour, clarity or the like, of polypropylene. In that regard, it turned out that also a filler may have an impact on turbidity formation. Thus, the absence of a filler may further contribute to reduction of turbidity formation.

In a further embodiment of the present disclosure, the polypropylene material is free of a neutralizer, in particular hydrotalcite (Mg₆Al₂[(OH)₁₆CO₃]·4H₂O), aluminum magnesium carbonate hydroxide, calcium stearate or a mixture thereof. The term “neutralizer” as used according to the present disclosure means a material being capable of neutralizing an acidic aqueous media, in particular an acidic aqueous solution. In that regard, it further turned out that also a neutralizer may have an impact on turbidity formation. Thus, the absence of a neutralizer may further contribute to reduction of turbidity formation.

In a further embodiment of the present disclosure, the polypropylene material is free of dialkyl-dialkoxy silane compounds (R₂Si(OR)₂) such as cyclohexyl-dimethoxy-methylsilane. In that regard, it turned out that cyclohexyl-dimethoxy-methylsilane may generate cyclohexyl-dihydroxy-methylsilane being leachable in a diluent and contributing to turbidity formation. Thus, the absence of cyclohexyl-dimethoxy-methylsilane may further aid to lower turbidity formation.

Further, the polypropylene material may have a proportion of ethylene units of ≤2.5% by weight, preferably <2.4% by weight, in particular of 1.8% by weight to 2.3% by weight, based on the total weight of the polypropylene material.

Especially preferred, the polypropylene material is a polypropylene material commercially available under Lumicene® MR10MM0.

In a further embodiment of the present disclosure, the polypropylene material has a proportion of 50% by weight to 100% by weight, in particular 60% by weight to 100% by weight, in particular 65% by weight to 95% by weight, preferably 70% by weight to 90% by weight, for example 80% by weight, based on the total weight of the inner layer.

In a further embodiment of the present disclosure, the inner layer further comprises a modifier material.

The term “modifier material” as used according to the present disclosure means a material, in particular plastic material, which is able to alter or improve, i.e. decrease or increase, properties, in particular impact strength and/or flexibility and/or heat seal strength, of the polypropylene material.

In a further embodiment of the present disclosure, the modifier material comprises or consists of a polymer. The polymer may be particularly a thermoplastic elastomer. Preferably, the polymer is selected from the group consisting of styrene-ethylene-butylene-styrene (SEBS) copolymer, styrene-ethylene-butylene (SEB) copolymer, styrene-ethylene-styrene (SES) copolymer, styrene-butylene-styrene (SBS) copolymer, styrene-ethylene-propylene (SEP) copolymer, styrene-ethylene-butadiene-styrene copolymer and mixtures of at least two of the afore-said modifier materials.

More specifically, the modifier material preferably comprises or consists of a polymer, in particular thermoplastic elastomer, selected from the group consisting of styrene-ethylene-butylene-styrene (SEBS) block copolymer, styrene-ethylene-butylene (SEB) block copolymer, styrene-ethylene-styrene (SES) block copolymer, styrene-butylene-styrene (SBS) block copolymer, styrene-ethylene-propylene (SEP) block copolymer, styrene-ethylene-butadiene-styrene block copolymer and mixtures of at least two of the afore-said polymers.

Especially preferably, the modifier material comprises or consists of styrene-ethylene-butylene-styrene (SEBS) copolymer, in particular styrene-ethylene-butylene-styrene (SEBS) block copolymer.

In a further embodiment of the present disclosure, the inner layer comprises a further polypropylene material. Preferably, the further polypropylene material is a component of the modifier material. The further polypropylene material may be identical to or different from the previously described polypropylene material of the inner layer.

In a further embodiment of the present disclosure, the modifier material has a proportion of 0% by weight or >0% by weight to 40% weight, in particular 0% by weight or >0% by weight to 30% by weight, preferably 5% weight to 20% by weight, for example 20% by weight, based on the total weight of the inner layer. More preferably, the modifier material has a proportion of 5% by weight to 50% by weight, in particular 5% by weight to 40% by weight, preferably 5% by weight to 30% by weight, based on the total weight of the inner layer.

In a further embodiment of the present disclosure, the polymer, in particular thermoplastic polymer, of the modifier material has a proportion of 0% by weight or >0% by weight to 40% by weight, in particular 0% by weight or >0% by weight to 30% by weight, preferably 10% by weight to 20% by weight, based on the total weight of the modifier material.

In a further embodiment of the present disclosure, the further polypropylene material has a proportion of 50% by weight to 100% by weight, in particular 60% by weight to 100% by weight, or 0% by weight to 30% by weight, in particular 10% by weight to 30% by weight, based on the total weight of the modifier material.

In a further embodiment of the present disclosure, the inner layer has a surface wettability, in particular determined via a methanol ink test (available from Plasmatreat GmbH, Germany), ≤30 mN/m, preferably ≤29 mN/m, in particular of 23 mN/m to 30 mN/m or 23 mN/m to 29 mN/m. By performing the methanol ink test, a test ink is applied quickly to a surface of a substrate using a brush. It is started with an ink with a high surface tension (such as 72 mN/m) directly after the pretreatment. If the brush stroke edges are stable for two seconds, the surface is easily wettable. Then, the surface tension of the substrate is at least equal to the value of the test ink. If the brush strokes of the test ink contract, the next lower test ink should be used. This way, the surface tension value of the material of the substrate is gradually approached. The surface tension of the material is equal to the value of the test ink last used that showed good wetting for at least 2 seconds. The test inks applied in the methanol ink test are manufactured according to DIN Draft 53364 or ISO 8296.

In a further embodiment of the present disclosure, the inner layer has a surface roughness as Roughness Average (Ra), in particular determined via confocal microscopy, preferably confocal laser scanning microscopy (CLSM) or laser scanning confocal microscopy (LSCM), of 0.05 Ra (μm) to 0.20 Ra (μm), in particular 0.06 Ra (μm) to 0.18 Ra (μm), preferably 0.08 Ra (μm) to 0.15 Ra (μm). A low surface roughness of the inner layer is advantageous, in particular in terms of a low or lower surface area. Thus, less active impurities, in particular metal impurities, may be presented on the surface of the inner layer which in turn helps to reduce interactions of the active pharmaceutical ingredient with the surface of the inner layer which may otherwise result, for example, in agglomeration, oligomerization such as dimerization and/or decomposition of the active pharmaceutical ingredient. This also contributes to reduction of turbidity formation.

In a further embodiment of the present disclosure, the inner layer has a thickness of 10 μm to 180 μm, in particular 10 μm to 150 μm, preferably 10 μm to 50 μm, for example 40 μm. The inner layer may be particularly in the form of an inner film layer.

In a further embodiment of the present disclosure, the wall is a multi-layered wall, i.e. a wall comprising or consisting of two or more, for example three, four or five, layers. Preferably, the wall is a three-layered wall comprising the inner layer, a middle layer and an outer layer.

The term “middle layer” as used according to the present disclosure means a layer which is arranged, in particular directly, i.e. immediately, between the inner layer and the outer layer of the three-layered wall.

The term “outer layer” as used according to the present disclosure refers to a layer which separates or defines the medical container from its environment or surroundings.

The middle layer may also comprise a polypropylene material. The polypropylene material of the middle layer may be identical to or different from the polypropylene material of the inner layer and/or a polypropylene material of the outer layer. Further, the middle layer may also comprise a modifier material. The modifier material of the middle layer may be identical to or different from the modifier material of the inner layer and/or a modifier material of the outer layer. Further, the middle layer may be in particular in the form of a middle film layer.

The outer layer may also comprise a polypropylene material. The polypropylene material of the outer layer may be identical to or different from the polypropylene material of the inner layer and/or middle layer. Further, the outer layer may also comprise a modifier material. The modifier material of the outer layer may be identical to or different from the modifier material of the inner layer and/or middle layer. Further, the outer layer may be in particular in the form of an outer film layer.

Further preferred, the inner layer and/or middle layer and/or outer layer are/is formed via extrusion, in particular cast extrusion and/or blown-film extrusion. Preferably, the inner layer and/or middle layer and/or outer layer are/is formed via cast extrusion. Blown-film extrusion has principally the advantage that it is cleaner regarding particulate contamination and results in a lower surface roughness, while cast extrusion exhibits the advantage that volatile compounds may evaporate more easily along a cast line.

As already mentioned, the medical container according to the present disclosure is preferably for the storage of a solution, preferably an aqueous solution of an active pharmaceutical ingredient. The solution may be preferably in the form of a ready-to-use solution of the active pharmaceutical ingredient, i.e. a directly available or pre-mixed or reconstituted solution of the active pharmaceutical ingredient, i.e. a solution of the active pharmaceutical ingredient which is ready for use and may be administered without any further preparation or mixing steps.

Alternatively, the medical container may be for the storage of an active pharmaceutical ingredient, in particular in powder form, and/or a liquid diluent, in particular for the active pharmaceutical ingredient, in particular wherein the active pharmaceutical ingredient and the liquid diluent are spatially separated from each other.

In a further embodiment of the present disclosure, the medical container contains an active pharmaceutical ingredient and/or a liquid diluent, particularly for the active pharmaceutical ingredient, in particular wherein the active pharmaceutical ingredient and the liquid diluent are spatially separated from each other. Typically, the liquid diluent is water. Further, the liquid diluent may comprise a chelating agent, in particular disodium salt of ethylene diamine tetra-acetic acid (disodium EDTA) and/or sodium citrate. Thus, the impact of metal impurities may be minimized.

In a further embodiment of the present disclosure, the medical container contains a solution, preferably an aqueous solution, containing an active pharmaceutical ingredient. The solution may be preferably in the form of a ready-to-use solution containing the active pharmaceutical ingredient.

In a further embodiment of the present disclosure, the active pharmaceutical ingredient is at least one antibiotic, in particular selected from the group consisting of piperacillin, tazobactam, cefazolin, meropenem, ampicillin, sulbactam, apramycin, neomycin, paromycin, spectinomycin, chloramphenicol, dirithromycin, erythromycin, doxycycline, tetracycline, linezolid, bacitracin, fosfomycin, fosmidomycin, ampicillin, amoxicillin, cloxacillin, pharmaceutically acceptable salts thereof and mixtures of at least two of the afore-mentioned antibiotics.

Preferably, the at least one antibiotic comprises piperacillin or a pharmaceutically acceptable salt thereof and/or tazobactam or a pharmaceutically acceptable salt thereof. More preferably, the at least one antibiotic is a combination or mixture of piperacillin and tazobactam or a combination or mixture of a pharmaceutically acceptable salt of piperacillin and a pharmaceutically acceptable salt of tazobactam.

Further, the medical container is preferably in sterilized form.

Further features and advantages of the present disclosure will become clear from the following description of preferred embodiments in form of figures, figure descriptions and examples. The individual features can be realized either singularly or severally in combination in one embodiment of the present disclosure. The preferred embodiments merely serve for illustration and better understanding of the present disclosure and are not to be understood as in any way limiting the present disclosure.

### BRIEF DESCRIPTION OF THE DRAWINGS

The Figures schematically show the following:

FIG. 1 shows an embodiment of a wall of a flexible medical container according to the present disclosure,

FIG. 2 shows an embodiment of a three-layered wall of a flexible medical container according to the present disclosure,

FIG. 3 shows an embodiment of a flexible medical container according to the present disclosure, and

FIG. 4 shows an exploded perspective view of the flexible medical container shown in FIG. 3, and

FIG. 5 shows surface roughness of inner layers in different medical containers.

### DETAILED DESCRIPTION

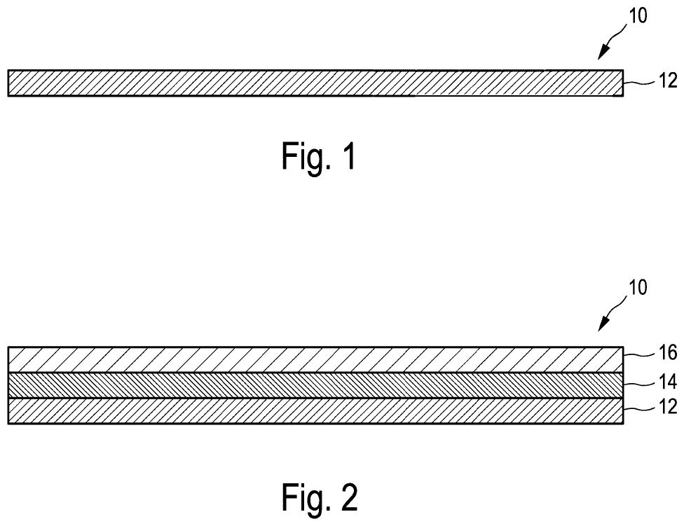

FIG. 1 schematically displays an embodiment of a wall 10 of a medical container according to the present disclosure.

The wall 10 comprises an inner layer 12 being configured or adapted to encase or surround, in particular directly, an active pharmaceutical ingredient, in particular a solution, preferably an aqueous solution, of an active pharmaceutical ingredient.

The inner layer 12 comprises a polypropylene material. Preferably, the polypropylene material is produced or synthesized via a metallocene catalyst. The metallocene catalyst may have particularly the formula I as disclosed in the general description. Preferably, the metallocene catalyst is free of titanium. In particular, the metallocene catalyst comprises zirconium as transition metal.

More preferably, the polypropylene material of the inner layer 12 may be free of titanium, in particular elemental titanium or titanium containing compounds, typically with the titanium having an oxidation state of +4. Thus, any effect of titanium on turbidity formation may be advantageously avoided.

Further, the polypropylene material may have an amount of aluminum, in particular elemental aluminum or aluminum containing compounds, typically with the aluminum having an oxidation state of +3, <80 μm/g.

Further, the polypropylene material of the inner layer 12 may preferably have a polydispersity index of 1 to 4, in particular 2 to 3, preferably 2.5 to 3. For example, the polypropylene material may have a polydispersity index of 2.8. In that regard, it surprisingly turned out that a more homogeneous distribution of polymer chains of the polypropylene material resulted in a smoother surface of the inner layer 12. A smoother surface in turn means a reduced surface area, and thus less metal or other element impurities, in particular of titanium and/or aluminum and/or silicon, which may foster undesired interactions between the active pharmaceutical ingredient and the inner surface 12. Such undesired interactions in turn may result in aggregation, oligomerization and/or decomposition such as hydrolysis of the active pharmaceutical ingredient which may increase turbidity formation.

Further, the polypropylene material of the inner layer 12 is preferably free of a filler such as synthetic silica, and/or a neutralizer such as hydrotalcite and/or aluminum magnesium carbonate hydroxide. Thus, any adverse effect of fillers and/or neutralizers on turbidity formation may be advantageously avoided.

Further, the polypropylene material of the inner layer 12 is preferably free of cyclohexyl-dimethoxy-methylsilane. Thus, any leaching of cyclohexyl-dihydroxy-methylsilane being generated from cyclohexyl-dimethoxy-methylsilane and promoting turbidity formation may be circumvented.

The polypropylene material of the inner layer 12 may be particularly a polypropylene material that is commercially available under PP Lumicene® MR10MM0.

In particular, the polypropylene material may have a proportion of 50% by weight to 100% by weight, in particular 60% by weight to 100% by weight, for example 80% by weight, based on the total weight of the inner layer 12.

The inner layer 12 may further, i.e. along the polypropylene material, comprise a modifier material. The modifier material preferably comprises a thermoplastic elastomer, in particular styrene-ethylene-butylene-styrene (SEBS) copolymer. Further, the inner layer 12 may comprise a further polypropylene material being a component of the filler material. The further polypropylene material may be identical to or different from the previously described polypropylene material of the inner layer 12.

The modifier material may have a proportion of 5% by weight to 50% by weight, in particular 5% by weight to 30% by weight, for example 20% by weight, based on the total weight of the inner layer 12.

Further, the thermoplastic elastomer may have a proportion of 0% by weight or >0% by weight to 40% by weight, in particular 0% by weight or >0% by weight to 30% by weight, based on the total weight of the modifier material.

Further, the further polypropylene material may have a proportion of 10% by weight to 30% by weight, based on the total weight of the modifier material.

Further, the inner layer 12 may have thickness from 10 μm to 180 μm, for example of 40 μm. The inner layer 12 may be particularly in the form of an inner film layer.

Further, the inner layer 12 preferably has a comparable low surface wettability. More specifically, the inner layer 12 may have a surface wettability, in particular determined via a methanol ink test, ≤30 mN/m. A lower surface wettability advantageously reduces interaction possibilities between the surface of the inner layer 12 and the active pharmaceutical ingredient and therefore lowers the risk that the active pharmaceutical ingredient may be absorbed onto the surface of the inner layer 12. This in turn reduces the risk of aggregation, oligomerization such as dimerization and/or decomposition of the active pharmaceutical ingredient, thereby contributing to a lower turbidity formation.

Further, the inner layer 12 preferably has a comparable low surface roughness. More specifically, the inner layer 12 may have a surface roughness as Roughness Average (Ra), in particular determined via confocal microscopy, from 0.06 Ra to 0.18 Ra. A lower surface roughness means a lower surface area, and therefore in particular less impurities exposed on the surface of the inner layer 12 which may foster undesired interactions between the impurities and the active pharmaceutical ingredient which would result in a higher turbidity formation.

FIG. 2 schematically shows an embodiment of a three-layered wall 10 of a flexible medical container according to the present disclosure.

The wall 10 comprises an inner layer 12. With respect to the features and advantages of the inner layer 12, reference is made in its entirety to the description of FIG. 1. The features and advantages described in terms of the inner layer 12 in the context of FIG. 1 do apply, mutatis mutandis, with respect to the inner layer 12 of the wall 10 according to FIG. 2.

The wall 10 further comprises a middle layer 14 and an outer layer 16.

The middle layer 14 may be directly arranged between the inner layer 12 and the outer layer 16. The middle layer 14 may also comprise a polypropylene material and in particular a modifier material. The polypropylene material of the middle layer 14 may be identical to or different from the polypropylene material of the inner layer 12 and/or a polypropylene material of the outer layer 16. The modifier material of the middle layer 14 may be identical to or different from the modifier material of the inner layer 12 and/or a modifier material of the outer layer 16. In particular, the middle layer 14 may be in the form of a middle film layer.

The outer layer 16 may be directly arranged onto the middle layer 14. The outer layer 16 separates or defines the medical container from its environment or surroundings. The outer layer 16 may also comprise a polypropylene material and in particular a modifier material. The polypropylene material of the outer layer 16 may be identical to or different from the polypropylene material of the inner layer 12 and/or middle layer 14. The modifier material of the outer layer 16 may be identical to or different from the modifier material of the inner layer 12 and/or middle layer 14. In particular, the outer layer 16 may be in the form of an outer film layer.

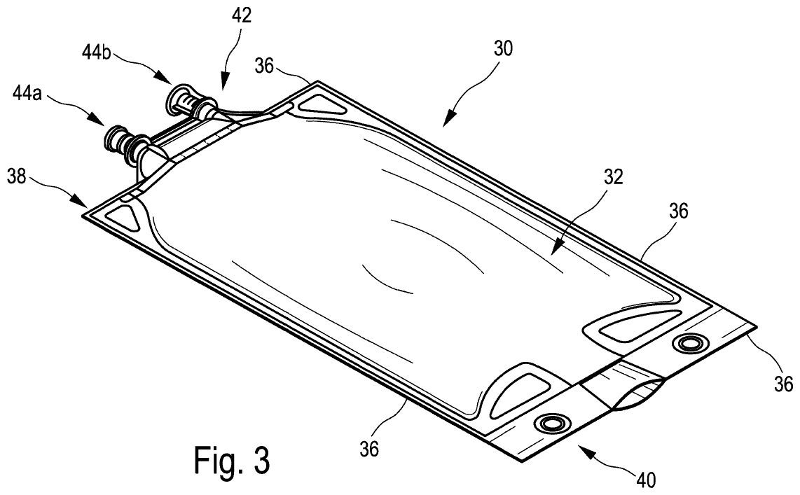

FIG. 3 schematically displays an embodiment of a medical container 30 according to the present disclosure.

The medical container 30 is in the form of a flexible medical container, in particular in the form of a medical bag, and comprises a first or front sheet 32 and a second or back (rear) sheet 34. The first sheet 32 and/or second sheet 34 have a wall comprising an inner layer as shown in FIG. 1 or a three-layered wall as shown in FIG. 2.

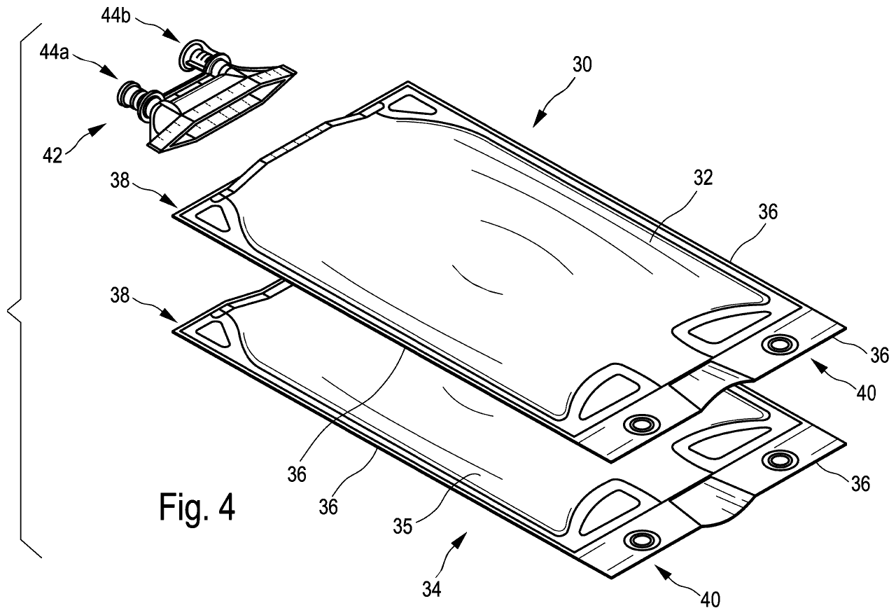

The sheets 32, 34 forming the medical container 30 can be provided separately, as shown in FIG. 4, and sealed together along and/or near their peripheral edges 36. Alternatively, the sheets 32, 34 can be formed from a single sheet comprising an inner wall as shown in FIG. 1 or a multi-layered wall as shown in FIG. 2, which is subsequently folded-over and sealed, for example via heat seal which extends substantially around the peripheral portion of the medical container 30. The sealed-together sheets 32, 34 form a body or cavity of the container 30.

Further, the two sheets 32, 34 can be welded together to form compartments within the body of the container 30. For example, one compartment can be filled with an active pharmaceutical ingredient in powder form and another compartment can be filled with a liquid diluent.

Alternatively, the flexible medical container 30 may comprise only one cavity or compartment, which may be filled with or contains an active pharmaceutical ingredient, in particular a solution of an active pharmaceutical ingredient.

Further, the sheets 32, 34 may be transparent. This advantageously allows the contents of the container 30 to be visually inspected, for example to allow a level of a solution of the active pharmaceutical ingredient to be seen, either during dispensing or prior to dispensing to ensure that the active pharmaceutical ingredient has not undergone a visually recognizable degradation, oligomerization and/or decomposition such as hydrolysis or the like, which would be indicated by an increased turbidity formation.

Further, the medical container 30 may have a top end 38 and a bottom end 40 and a port end cap 42, which is attached to or within the top ends 38 of the first sheet 32 and second sheet 34 such that one or more port assemblies 44a, 44b are in fluid communication with at least one internal compartment (not shown) of the body and cavity, respectively, of the flexible medical container 30.

### Example Section

### 1. Compilation of Data

The below Table 1 compiles data on catalyst structure, metal impurities, selected polypropylene properties and turbidity in containers for storing piperacillin and tazobactam (Pip-Taz) to be administered intravenously.

TABLE 1

| | Lumicene ® | | |
|---|---|---|---|
| Type of | MR10MM0 | PP Z 9450 | PP 7450 HC |
| polypropylene | (B. Braun part No. | (B. Braun Part No. | (B. Braun Part No. |
| material | X52-828) | X52-916) | X52-921) |
| type of catalyst | metallocene | Ziegler-Natta | Ziegler-Natta |
| filler | — | — | synthetic silica |
| neutralizer | — | hydrotalcite, | Hydrotalcite, |
| | | aluminum | aluminum |
| | | magnesium carbonate | magnesium carbonate |
| | | hydroxide | hydroxide |
| number average | 73.000 | 42.000 | 43.000 |
| molecular weight | | | |
| (Mn)/GPC | | | |
| weight average | 204.000 | 292.000 | 305.000 |
| molecular weight | | | |
| (Mw)/GPC | | | |
| polydispersity index | 2.8 | 7.0 | 7.1 |
| (Mw/Mn) | | | |
| proportion of | 2.0 | 6.1 | 3 |
| ethylene units | | | |
| Pip-Taz turbidity in | 1.86 | 13.40 | 24.20 |
| IV container | (in BFlex Film-80% | (in Duplex III clear | (in excel overwrap |
| (4 weeks at 40° C.) | of | film- | with |
| | Lumicenc-20% | 80% of PP Z9450- | 100% of PP7450) |
| | SBESs) | 20% | |
| | | Kraton GI652) | |
| Pip-Taz turbidity | 1.39 | 4.55 | 2.08 |
| with PP resins | | | |
| (3 weeks at 40° C.) | | | |

The polypropylene materials being commercially available under PP Z 9450 and PP 7450 HC comprise the largest amount of aluminum which is due to the fact that these polymeric materials were synthesized in the presence of aluminum containing Ziegler-Natta types of catalysts. The main material difference in the production between the polypropylene PP 7450 HC and PP Z 9450 is that 2.000 ppm silica filler was used in the polypropylene material PP 7450 HC. Cyclohexyl-dimethoxy-methylsilane amount used in the polypropylene material PP 7450 HC is less than that used in the polypropylene material PP Z 9450. Cyclohexyl-dimethoxy-methylsilane generates cyclohexyl-dihydroxy-methylsilane leachable in sodium chloride diluent solution during storage in a container.

Cyclohexyl-dihydroxy-methylsilane leachable may contribute to turbidity formation. Synthetic silica in the polypropylene material PP 7450 HC may also contribute to turbidity.

It turned out that the polypropylene material commercially available under Lumicene® MR10MM0 has the lowest amount of total residual metal impurities, does not have titanium, and does not have a filler or neutralizer of any kind. Turbidity in a container constructed with a polypropylene material being commercially available under Lumicene® MR10MM0 has the lowest amounts of turbidity (Notebook BB5254/137). As it can be concluded, the polypropylene materials commercially available under PP 7450 HC and PP Z 9450 have the highest amounts of the residual metal impurities, including titanium and aluminum, have neutralizer and/or filler and have cyclohexyl-dimethoxy-methylsilane as process aid which will have cyclohexyl-dihydroxy-methylsilane leachable (a silicon-containing leachable) in a diluent chamber of the respective containers.

The direct contact study of piperacillin and tazobactam with the different polypropylene materials showed turbidities from higher to lower as follows:

$$PP Z 9450 > PP 7450 HC > {Lumicene}^{®} MR 10 MM 0.$$

Polydispersity index (Mw/Mn) of the three polymer materials also correlates to turbidity results: narrow molecular weight distribution (smaller polydispersity index) correlates to less turbidity. Mn is the number average molecular weight, and Mw is the weight average molecular weight. The midpoint of the distribution in terms of the number of polymerized molecules is Mw. The largest differences between Mw and Mn numbers as polydispersity index was observed for PP Z 9450 at 7.0 and PP 7450 HC at 7.1, respectively, while polydispersity index of Lumicene® MR10MM0 is at 2.8. The polymeric materials PP Z 9450 and PP 7450 HC have more diverse short chains and long-chain polypropylene molecules and create a rougher surface. The polymeric material Lumicene® MR10MM0 has the smallest difference between Mw and Mn, and thus has a more homogenous distribution of polymer chains, and potentially creating smoother polymeric surface, in particular polymer film surface.

### 2. Impact of Elemental Impurities and Container Leachables

In order to mimic and evaluate the impact of Ti, Al, Sn, Ni, Mg, Cr, and Si containing impurities on the turbidity of piperacillin and tazobactam formation, the following simulating experiments were carried out (reference 6.7, Notebook BB5254/125, 134, 140-141, 145-146). Powders of piperacillin and tazobactam were mixed with different potential impurities in glass vials and incubated at 40° C. for 3 weeks. The powders were reconstituted in 0.9% aqueous sodium chloride solution prior to turbidity analysis. The results are summarized in the below Table 2.

TABLE 2
Turbidity Results 40° C. for 3 weeks

| | | Drug & | | |
|---|---|---|---|---|
| | | Chemicals | | |
| | | (Target Mole | | |
| | | Ratio = | | |
| | | 100:1, ~100 ppm | Chemical only | |
| | Description | for chemicals) | (~100 ppm) | Delta |
| 0 | drug only (control) | 1.10 | N/A | 1.10 |
| 1 | nickel acetate | 1.14 | 0.22 | 0.92 |
| | tetrahydrate | | | |
| 2 | chromium (Ill) | 15.60 | 0.18 | 15.42 |
| | chloride | | | |
| | hexahydrate | | | |
| 3 | tin (IV) acetate | 1.13 | 0.08 | 1.04 |
| 4 | iron(ll) acetate | 98.77 | 9.66 | 89.10 |
| 5 | aluminum chloride | 3.01 | 0.13 | 2.88 |
| | hydrate | | | |
| 6 | titanium (IV) | 179.33 | 5.44 | 173.89 |
| | oxyacetylacetonate | | | |
| 7 | Cyclohexyl- | 18.20 | 0.54 | 17.66 |
| | dihydroxyl- | | | |
| | hydroxylsilane | | | |
| | (CDHS) | | | |
| 8 | magnesium | 1.53* | N/A* | N/A* |
| | sulfate* | | | |

Study Procedure & Condition:
time frame: 3 weeks at 40° C.
0.3 g Pip-Taz with chemicals of- 1-2 mg in a glass vial (estimated drug to chemicals molar ratio: 100/1)
after the incubation, reconstituted the powers in 12 mL of 0.9% NaCl for turbidity analysis.
study was performed respectively with drug/chemical ratio of −1:1 and chemical concentration of 5450 ppm. N/A: No control group available

As it can be concluded from the above Table 2, not all metal cations can create turbidity in case of a solution of piperacillin and tazobactam. For example, Ni and Mg have no impact on the turbidity of piperacillin and tazobactam. There is some selectivity toward the nature of the metal impurity: Ti, Al, and Si all have a substantial effect on turbidity formation. Ti may even have a catalytic effect, since from the “zero” time when the starting ingredient was mixed larger amounts of the precipitate were detected. The presence of the Si compounds also has a measurable effect on turbidity formation.

In summary, the simulated experiments confirm that the presence of Ti and Al can result in turbidity formation of piperacillin and tazobactam. This may be in particular due to the fact that said heavy metals may induce surface oxidation and change properties of a layer, in particular film, made of the respective polymeric material.

The cyclohexyl-dihydroxy-methylsilane as leachable from the polypropylene material PP Z 9450 also contributed to the turbidity formation as shown in Table 2. The cyclohexyl-dihydroxy-methylsilane concentration of around 200 μg/ml was used in Table 2. The comparison of turbidity from Duplex III container to study in Table 2 with a concentration of cyclohexyl-dihydroxy-methylsilane is listed in Table 3 below.

TABLE 3

| | | | | | Turbidity | Ratio of |
|---|---|---|---|---|---|---|
| | | | | Ratio of | at 40″C | Turbidity |
| | | Volume of | CDHS | CDHS to | for 3 | to Ratio |
| Data | Pip-Taz | 0.9% NaCl | concentration | Pip-Taz | weeks | of (CDHS/ |
| Reference | used (g) | (ml) | (μg/ml) | (μg/g) | (NTU) | Pip-Taz) |
| RPT-RD- | 3.375 | 56.5 | 15 (estimated | 251 | 6.432 | 0.026 |
| 1000172 | | | based on | | | |
| (reference | | | RPT-PH-1 | | | |
| 6.5 and 6.7) | | | 012477, | | | |
| | | | Reference 6.8) | | | |
| Table 2 | 0.5 | 12 | 200 | 4800 | 18.2 | 0.004 |

The data from Table 3 showed that the ratio of turbidity to the ratio of cyclohexyl-dihydroxy-methylsilane/piperacillin and tazobactam from Duplex III container stored piperacillin and tazobactam is much higher than that by using cyclohexyl-dihydroxy-methylsilane in glass container. Therefore, cyclohexyl-dihydroxy-methylsilane contributed to the portion of turbidity formation.

### 3. Impact of Turbidity Surface Roughness and Surface Wettability

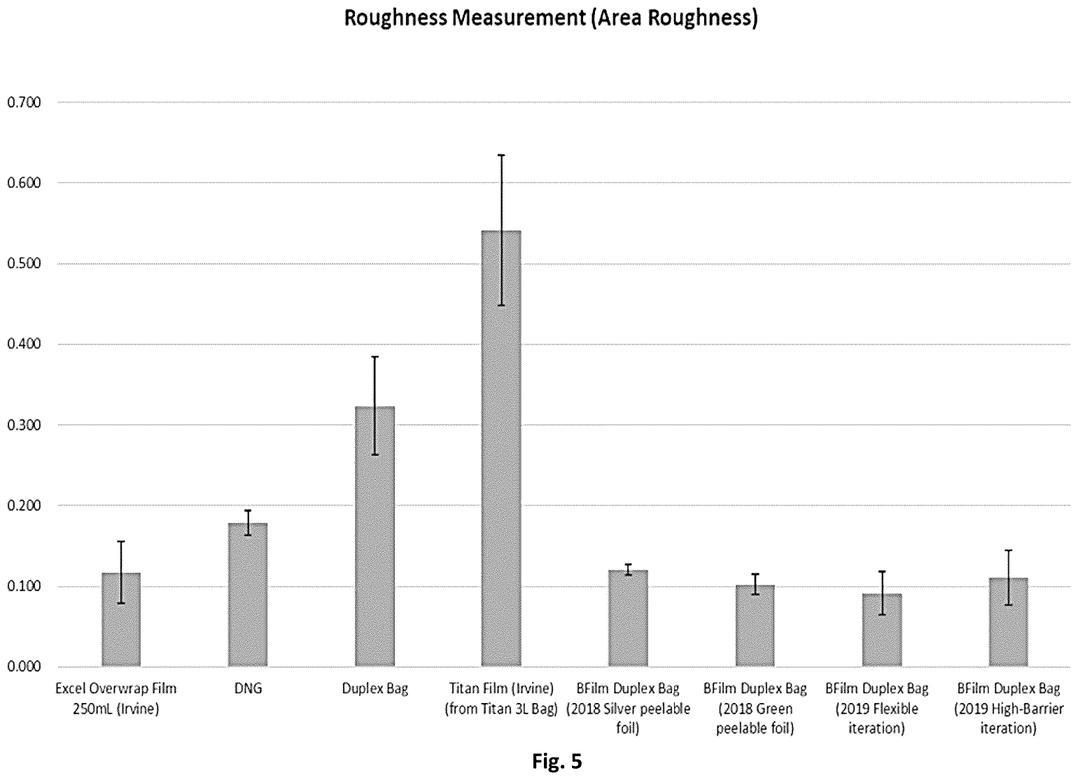

It has been observed that the surface roughness of the BFlex Duplex container is constantly lower and surface roughness in Duplex film is higher. It can be attributed by the material properties used to fabricate the container, such as polydispersity index (Mw/Mn) at 7.0 for the polypropylene material Z 9450 in Duplex III and at 2.8 for the polypropylene material Lumicene® MR10MM0 in BFlex Duplex, respectively. The surface roughness of different layers and films, respectively, is shown in FIG. 5.

In FIG. 5 the roughness results of Duplex III (20% Kraton G 1652), titanium (40% Kraton G 1652), Excel Overwrap (0% Kraton G 1652) showed that the container contains the highest percentage of Kraton G 1652, the roughness will be higher. However, the turbidity from Excel Overwrap with 100% polypropylene material 7450 HC and lower roughness had higher turbidity in comparison with Duplex III with 20% Kraton G 1652 with higher roughness.

It has also been found that Duplex III layer and film, respectively, has higher surface wettability (46 mN/m) than that of BFlex Duplex film and layer, respectively (≤30 mN/m). Methanol test inks (B Series), suitable for all common surfaces, 30 to 72 mN/m (in increments of 2 mN/m) was used to determine the surface wettability. The results confirmed that the BFlex Duplex film and layer, respectively, resulted in considerably lower surface wettability.

A higher surface wettability indicates that the surface is more hydrophilic, which may influence absorption of piperacillin and tazobactam onto the film surface and layer surface, respectively, and thereby fosters aggregation to larger particles.

In fact, the particle sizes from different container systems or film surface and layers surface, respectively, properties showed that larger particle size of piperacillin and tazobactam stored was found in Duplex III container in comparison with BFlex Duplex container, which is correlated to the film or layer surface wettability.

Kinetically speaking, agglomeration, dimerization, and decomposition reactions of piperacillin and tazobactam may happen on the surface of the containers, where the solution of piperacillin and tazobactam is in contact with catalytic amounts of the active elements, e.g. Al, Ti, and Si derivatives. The rougher surface will result in more surface area with active impurities, which did not leach or be extracted into the solution. As a result, a smoother surface will help to decrease aggregation of piperacillin and tazobactam. This is the case of a smoother inner layer or film comprising the polypropylene material Lumicene® MR10MM0 in BFlex containers versus an inner layer or film comprising the polypropylene material Z 9450 in Duplex III containers. Related to this effect is the surface wettability of the layer and film, respectively. The presence of different chemical elements on the surface, for example, hydrophilic metal cations with the highest oxidation states of +4 and +3, e.g. Ti, Si, and Al, may help to increase the wettability of the surface by methanol and/or water, and coordination of electron-rich atoms of nitrogen and oxygen of molecules of piperacillin and tazobactam. Thus, promoting absorption of molecules of piperacillin and tazobactam to the surface promote the further aggregation process of piperacillin and tazobactam on the surface.

### 4. Power Aggregation Investigation of Piperacillin and Tazobactam

Microscope investigation was performed on powders consisting of a mixture of piperacillin and tazobactam after being stored in different types of containers (see the below Table 4).

TABLE 4

| | | | | B2 | B4 |
|---|---|---|---|---|---|
| | A1 | A3 | B1 | B. Film | API in Glass |
| | Duplex | Duplex | MiniSterBag | Duplex | Vial |
| | Highest | High | Medium | Low | Low |
| | turbidity: | turbidity: | turbidity: | turbidity: | turbidity: |
| Group | 22.20 | 12.30 | 2.88 | 1.17 | 0.92 |
| mean particle | 47.27 | 34.30 | 29.69 | 26.50 | 35.78 |
| size | | | | | |
| std. DV | 38.46 | 20.10 | 11.35 | 9.31 | 22.61 |
| max diameter | 196.35 | 132.98 | 62.76 | 61.03 | 108.80 |
| min diameter | 15.43 | 15.58 | 15.66 | 15.51 | 15.58 |

It was discovered that the particle size of piperacillin and tazobactam is larger, the turbidity of the reconstituted solution is higher. The largest size of particles was observed from the Duplex III container. It may be connected to kinetic effects with the polymer material inducing the starting of powder aggregation from container surface interaction.

1. A flexible medical container having a wall comprising an inner layer, wherein the inner layer comprises a polypropylene material made in the presence of a metallocene catalyst.

2. The flexible medical container according to claim 1, wherein the metallocene catalyst is a titanium-free metallocene catalyst.

3. The flexible medical container according to claim 1, wherein the polypropylene material is free of titanium.

4. The flexible medical container according to claim 1, wherein the polypropylene material has an amount of aluminum <80 μg/g.

5. The flexible medical container according to claim 1, wherein the polypropylene material has a polydispersity index (Mw/Mn) from 1 to 4.

6. The flexible medical container according to claim 1, wherein the polypropylene material is free of a filler.

7. The flexible medical container according to claim 1, wherein the polypropylene material is free of a neutralizer.

8. The flexible medical container according to claim 1, wherein the polypropylene material is free of cyclohexyl-dimethoxy-methylsilane.

9. The flexible medical container according to claim 1, wherein the polypropylene material has a proportion of 60% by weight to 100% by weight.

10. The flexible medical container according to claim 1, wherein the inner layer further comprises a modifier material.

11. The flexible medical container according to claim 10, wherein the modifier material comprises a polymer selected from the group consisting of styrene-ethylene-butylene-styrene (SEBS) copolymer, styrene-ethylene-butylene (SEB) copolymer, styrene-ethylene-styrene (SES) copolymer, styrene-butylene-styrene (SBS) copolymer, styrene-ethylene-propylene (SEP) copolymer, styrene-ethylene-butadiene-styrene copolymer and mixtures of at least two of the afore-said modifier materials.

12. The flexible medical container according to claim 11, wherein the polymer has a proportion of 0% by weight to 40% by weight.

13. The flexible medical container according to claim 10, wherein the inner layer comprises a further polypropylene material, wherein the further polypropylene material is a component of the modifier material.

14. The flexible medical container according to claim 13, wherein the further polypropylene material has a proportion of 50% by weight to 100% by weight.

15. The flexible medical container according to claim 10, wherein the modifier material has a proportion of 5% by weight to 50% by weight.

16. The flexible medical container according to claim 1, wherein the inner layer has a surface wettability of ≤30 mN/m.

17. The flexible medical container according to claim 1, wherein the inner layer has a surface roughness of 0.05 Ra to 0.20 Ra.

18. The flexible medical container according to claim 1, wherein the inner layer has a thickness of 10 μm to 180 μm.

19. The flexible medical container according to claim 1, wherein the wall is a multi-layered wall.

20. The flexible medical container according to claim 1, wherein the flexible medical container contains an active pharmaceutical ingredient.

21. The flexible medical container according to claim 20, wherein the active pharmaceutical ingredient comprises at least one antibiotic.
